SEQUENCE LISTING (19 sequences)

SEQ ID NO: 1 - DNA, 30 bp, Artifical Sequence
  Primer Sequence
tcgaggatcccccttattatcaagataaga

SEQ ID NO: 2 - DNA, 48 bp, Artificial Sequence
  Primer Sequence
taatgagtgagaattaattccaggttagtgacattagaaaaccgactg

SEQ ID NO: 3 - DNA, 48 bp, Artificial Sequence
  Primer Sequence
cagtcggttttctaatgtcactaacctggaattaattctcactcatta

SEQ ID NO: 4 - DNA, 28 bp, Artificial Sequence
  Primer Sequence
acgtctatggatcctgtttcctgtgtga

SEQ ID NO: 5 - DNA, 39 bp, Artificial Sequence
  Primer Sequence
tcaggaattcactagtggtaaggtaaacgccattgtcag

SEQ ID NO: 6 - DNA, 39 bp, Artificial Sequence
  Primer Sequence
ttaggaattcctcgaggcataaaccagccattgagtaag

SEQ ID NO: 7 - DNA, 42 bp, Artificial Sequence
  Primer Sequence
ggaattaatgatgtctagattagataaaagtaaagtgattaa

SEQ ID NO: 8 - DNA, 36 bp, Arificial Sequence
  Primer Sequence
accatcagcgaaaaaggttatgctgcttttaagacc

SEQ ID NO: 9 - DNA, 47 bp, Artificial Sequence
  Primer Sequence
ggaacgaaaggaggtatatacatatgtgcgaactcgatattttacac

SEQ ID NO: 10 - DNA, 30 bp, Artificial Sequence
  Primer Sequence
ttttttcagctgcacctttggtcaccaacg

SEQ ID NO: 11 - DNA, 30 bp, Artificial Sequence
  Primer Sequence
gttcatgaaaaactaaaaaaaatattgaca

SEQ ID NO: 12 - DNA, 30 bp, Artificial Sequence
  Primer Sequence
ccatcggaagctgtggtatggctgtgcagg

SEQ ID NO: 13 - DNA, 30 bp, Artificial Sequence
  Primer Sequence
gacaagatgtgtatccaccttaacttaatg

SEQ ID NO: 14 - DNA, 24 bp, Artificial Sequence
  Primer Sequence
attctactacttgcaccgactgcc

SEQ ID NO: 15 - DNA, 24 bp, Artificial Sequence
  Primer Sequence
accacgaccttcacctttagtaga

SEQ ID NO: 16 - DNA, 26 bp, Artificial Sequence
  Primer Sequence
tgtgcttcaaataatgacaatgactc

SEQ ID NO: 17 - DNA, 24 bp, Artificial Sequence
  Primer Sequence
caccaggcaatactagatcttcag

SEQ ID NO: 18 - DNA, 27 bp, Artificial Sequence
  Primer Sequence
tgcaacatttgataaccaatgcgtgga

SEQ ID NO: 19 - DNA, 27 bp, Artificial Sequence
  Primer Sequence
tcattctcctttttcgctcgaggatgt

(19) United States
(12) Patent Application Publication
(10) Pub. No.: US 2002/0119573 A1
(43) Pub. Date: Aug. 29, 2002

(54) Footprinting plasmid

(76) Inventors: Karen J. Shaw, Glen Ridge, NJ (US); Teresa Kenney, Arlington, MA (US); George H. Shimer JR., Boston, MA (US)

Correspondence Address: DARBY & DARBY P.C., 805 Third Avenue, New York, NY, 10022, US

(21) Appl. No.: 09/796,088

(22) Filed: Feb. 28, 2001

(51) Int. Cl. C12N 15/74; C12Q 1/68; C12N 1/21

(52) U.S. Cl. 435/473; 435/320.1; 435/252.3; 435/6

(57) ABSTRACT

The present invention provides plasmids for insertional mutagenesis in bacteria. Specifically, the invention provides plasmids comprising a transposition system including a transposon and a transposase, in which transposon insertion can be temporally regulated. The plasmids can be used to determine the relative importance of a particular gene in viability of the organism from which the gene is derived. The plasmids are particularly useful for large-scale screening of a multiplicity of genes.

### FIELD OF THE INVENTION

This invention relates to a novel plasmid which is useful for determining whether a gene of interest is important for growth of the organism from which the gene is derived.

### BACKGROUND OF THE INVENTION

Large scale sequencing of the genomes of many different organisms has yielded DNA nucleotide sequence information for thousands of different genes. For the most part, the function of these genes remains unknown. Powerful techniques are available for determining whether a gene product is essential for growth and/or viability.

Conventional techniques examine the effect of deleting or disrupting a gene, i.e., by so-called gene “knockout.” Using this technique, a mutation in the coding sequence of the gene is created by in vitro insertion of a non-coding segment or mutation into the coding sequence for that gene. The mutated gene is then introduced into the genome of the organism by a variety of techniques to replace the wild-type version of the gene, and the ability of the resulting mutant strain to grow under a variety of different physiological conditions is tested. Although this strategy is effective for analyzing the function of a small number of genes, it is impractical to simultaneously analyze the function of large numbers of genes.

Genetic footprinting has been used to identify genes that are important for fungal viability (Smith et al., 1995, Proc. Natl. Acad. Sci. USA 92:5479-6433; Smith et al., 1996, Science 274:2069; U.S. Pat. No. 5,612,180). In genetic footprinting, transposon-mediated insertional mutagenesis is used to insert a predetermined nucleic acid sequence randomly throughout the genome of a cell; this is followed by growth of the mutagenized culture over multiple generations. Finally, each gene of interest is evaluated to determine whether the mutagenized culture contains the transposon inserted into the gene. Genes that are important for viability do not tolerate transposon insertions, while genes that are dispensable or redundant are more likely to tolerate transposon insertions. While this method allows for the analysis of a multiplicity of fungal genes, there is a need for an efficient and economical method that allows the rapid analysis of the function of a multiplicity of bacterial genes.

### SUMMARY OF THE INVENTION

The present invention fulfills this need by providing novel bacterial plasmids that are used to determine whether a bacterial gene is important for viability of the cell from which it is derived. These plasmids, which are designated “footprinting plasmids”, comprise: (i) a transposon carrying a selectable marker gene; (ii) a gene encoding a transposase, which is operably linked to a regulatable promoter; and (iii) an environmentally sensitive bacterial origin of replication. Preferably, the transposon and transposase are derived from Tn10. In a further embodiment of the invention, the environmentally sensitive bacterial origin of replication is a Gram-positive origin of replication.

In another aspect, the invention provides methods for determining whether particular genes of interest are important for viability. The methods are carried out by the steps of:

(a) transforming a bacterial culture with a footprinting plasmid according to the invention;

(b) maintaining episomal replication of the plasmid and inducing random insertion of the transposon into the genome of the transformed cell by incubating the transformed culture under conditions in which the regulatable promoter is active, thereby producing a mutagenized culture (T0);

(c) subjecting the mutagenized culture to continuous logarithmic growth, under conditions in which episomal replication of the plasmid and insertion of the transposon are repressed;

(d) extracting genomic DNA from T0 and subsequent bacterial culture; and

(e) analyzing each gene of interest for the presence or absence of the transposon.

In a preferred embodiment, the mutagenized culture is subjected to selection for the drug resistance marker carried on the transposon for at least twenty-five (T25) generations.

Genes that are important for viability are not likely to exhibit transposon insertions, whereas genes that are non-essential or redundant are likely to exhibit transposon insertions.

### DESCRIPTION OF THE DRAWINGS

FIG. 1 is a schematic illustration of the pG139+ vector. In this vector, the transposase gene is under the control of the PXYL promoter containing a tetracycline repressor operator and the tetracycline repressor. The mini-Tn10 transposon contains a chloramphenicol resistance marker for selection in S. aureus and an outward reading PLAC promoter.

FIGS. 2-7 are graphic illustrations of the PCR-ABI Tn10 insertion profiles in the S. aureus genomic DNA. T represents the number of generations where T=0 is the point at which the episomal replication of the plasmid and insertion of the transposon are repressed. T15 and T25 represent fifteen and twenty-five generations of growth following T0 under selective conditions for transposon insertions. TN represents a non-mutagenized culture.

FIGS. 2A-D show the Tn10 profile of a known non-essential S. aureus gene, abcA.

FIGS. 3A-D show the Tn10 profile of a known non-essential S. aureus gene, agrB.

FIGS. 4A-D show the Tn10 insertion profile of the proposed essential S. aureus gene,ftsZ.

FIGS. 5A-D show the Tn10 insertion profile of the proposed essential S. aureus gene, murD.

FIGS. 6A-D show the Tn10 insertion profile of the an unidentified non-essential S. aureus gene.

FIGS. 7A-D show the Tn10 insertion profile of an unidentified essential S. aureus gene.

### DETAILED DESCRIPTION OF THE INVENTION

All patents, patent applications, publications and other materials cited herein are hereby incorporated by reference in their entirety. In case of inconsistencies, the present description, including definitions, is intended to control.

Definitions

1. “Nucleic acid” or “polynucleotide” as used herein refers to purine- and pyrimidine-containing polymers of any length, either polyribonucleotides or polydeoxyribonucleotides or mixed polyribo-polydeoxyribo nucleotides. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases.

2. A “plasmid” as used herein refers to a circular or linear DNA vector that is capable of transforming a bacteria and is capable of replicating episomally in the transformed bacteria.

3. “Episomal” as used herein refers to a nucleic acid that replicates and is maintained extrachromosomally.

4. A “complement” of a nucleic acid sequence as used herein refers to an “antisense” nucleic acid sequence that participates in Watson-Crick base-pairing with an original nucleic acid sequence.

5. “Transposase” as used herein refers to an enzyme that catalyzes the movement of a transposon from one segment of DNA to another.

6. “Transposon” as used herein refers to a defined segment of DNA that is capable of moving from one region of DNA to another.

7. A “promoter” as used herein refers to a segment of DNA capable of activating transcription of a DNA segment to which it is operably linked.

8. “Log-phase” as used herein refers to a period in which exponential growth occurs.

9. “Polymerase Chain Reaction” (PCR) is a method for amplifying a nucleic acid sequence using two oligonucleotide primers (Saiki et al., 1988, Science 239:48).

10. A gene that is “important for viability” as used herein refers to a gene which, when altered, impairs viability, growth, and/or reproduction to a detectable extent under at least one growth condition. A gene that is “essential” is one that without which, the organism cannot survive.

11. “Environmentally sensitive bacterial plasmid origin of replication” as used herein refers to a bacterial origin of replication whose replication activity is regulated by environmental conditions. In a preferred embodiment, replication is regulated by temperature.

12. “Environmental conditions” include, but are not limited to, temperature, ion concentrations, available oxygen, glucose (or other sugar) level, nutrient or amino acid content, salt content, and pH.

13. “Antibiotic resistance gene” as used herein refers to a positive selection marker that eliminates or retards the effects of antibiotics (i.e. bacterial cell death). The term “antibiotic”, as used herein, refers to a chemotherapeutic agent that is capable of killing or inhibiting the growth of a microorganism. Antibiotics include, but are not limited to, ampicillin, cephalosporine, chloramphenicol, clindamycin, erythromycin, gentamycin, kanamycin, lincomycin, methicillin, mupirocin, polymyxins, quinolones, rimfampicin, spectinomycin, streptomycin, tetracyclines, and trimethoprin. In a preferred embodiment, the antibiotic is chloramphenicol.

The present invention provides novel nucleic acid vectors for use in genomic footprinting in bacteria. These vectors comprise: (i) a transposon carrying a selectable marker gene; (ii) a gene encoding a transposase operably linked to a regulatable promoter; and (iii) an environmentally sensitive bacterial origin of replication.

Genomic footprinting using the plasmids of the invention, allows for the rapid and efficient determination of the role in cell viability of a large number of genes. This methodology involves: (i) insertional mutagenesis using a transposon that inserts randomly at multiple sites throughout the genome of the cells; (ii) growth of the cells over multiple generations under selective condition for the transposon at the non-permissive temperature for plasmid replication; and (iii) determination of whether the transposon has inserted into the coding sequence of a gene of interest (Smith et al., 1995, Proc. Natl. Acad.Sci. USA 92:6479).

The environmentally sensitive bacterial origin of replication is regulated by external or environmental conditions such as, but not limited to, temperature and ion concentration (Hamilton et al., 1989, Journal of Bacteriology, 171:4617; Maguin et al., 1992, Journal of Bacteriology, 174:5633; Villfane et al., 1987, Journal of Bacteriology, 169:4822). In a preferred embodiment, the environmentally sensitive bacterial origin of replication is temperature sensitive. The environmentally sensitive bacterial origin of replication may be derived from a Gram-positive or a Gram-negative bacteria. Preferably, the bacterial origin of replication is derived from a Gram-positive bacterial strain. More preferably, sequences comprising such a bacterial origin of replication include, without limitation, sequences derived from pE194 (Villafane et al., 1987, J. of Bacteriology 169:4822-4829). The plasmids of the invention may be used in any Gram-negative and Gram-positive bacteria. Non-limiting examples of such bacteria include, but are not limited to, Salmonella typhimurium, Acinetobacter baumannii, Bacteroides fragilis, Enterobacter cloacae, Klebsiella pneumoniae, Moraxella catarrhalis, Proteus mirabilis, Pseudomonas aeruginosa, Campylobacter jejuni, Neisseria meningitides, Chlamyda pneumoniae, Chlamydia trachomatis, Escherichia coli, Haemophilus influenzae, Helicobacter pylori, and Rickettsia prowazekii. Preferred are Gram-positive strains. Non-limiting examples of such bacteria include, but are not limited to, Micrococcus luteus, Micrococcus roseus, Micrococcus varians, Staphylococcus aureus, Staphylococcus epidermidis, Streptococcus pyogenes, Streptococcus agalactiae, Enterococcus faecalis, Enterococcus faecium, Streptococcus pneumoniae, Streptococcus mutans, Streptococcus mitis, Bacillus anthracis, Bacillus cereus, Listeria monocytogenes, Listeria ivanovii, Erysipelothrix rhusiopathiea, and Corynebacterium diphtheriae. More preferably, the plasmids of the present invention are used in S. aureus bacteria.

When the environmentally sensitive origin of replication is a Gram-positive origin of replication, a second Gram-negative origin of replication may be incorporated into the plasmid of the invention in order to provide for the ability to replicate in Gram-negative bacteria such as, for example, E. coli. Sequences comprising such a bacterial origin of replication include, without limitation, sequences derived from pBR322. (Balbas et al., 1986, Gene 50:3-40)

Transposons for use in the plasmids of the invention encompass any sequence capable of randomly inserting into a bacterial genome, such as, e.g., sequences derived from Tn5, Tn7, Tn10, Tn551, Tn552, Tn554, Tn916, Tn917, Tn4001, and Tn1545. In a preferred embodiment, the transposon is Tn10. Transposons carry a selectable marker gene. The gene may be any gene that allows for identification of cells that incorporate the transposon of the present invention. In a specific embodiment, the selectable marker is any antibiotic resistance gene.

An antibiotic resistance gene is a positive selection marker that confers cellular immunity ti antibiotics present in the cellular system, under appropriate environmental conditions. Non-limiting examples of such antibiotic resistance genes includes amp and cat1.

Transposases for use in the invention encompass enzymes capable of catalyzing the random insertion of a transposon into a bacterial genome. For each transposon, there is a corresponsding transposase. A suitable transposase includes without limitation Tn10ATS. Suitable regulatable promoters to which the transposase may be operably linked include, without limitation, those derived from the PXYL promoter which contains a copy of the tertracycline repressor operator (Geissendorfer and Hillen, 1990, Applied Microbiology and Biotechnology, 33:657-663; Hillen et al., 1989, J. of Bacteriology, 171:3840-3845).

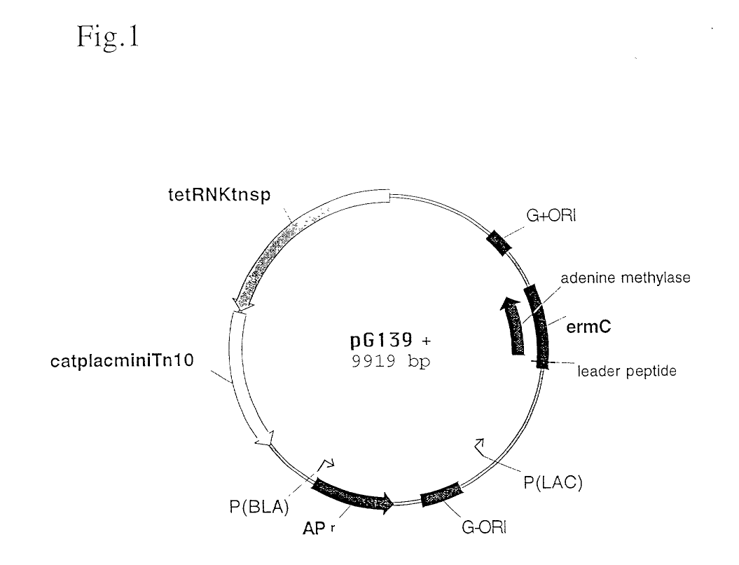

In a preferred embodiment, the plasmid comprises a transposase and transposon derived from Tn10; an ampicillin resistance gene; a pBR-derived origin of replication for propagation in E. coli; a pE194-derived temperature sensitive origin of replication for propagation in S. aureus; an erythromycin resistance gene for selection in S. aureus; a chloramphenicol resistance gene within the transposon; and a transposase gene under the control of a regulatable promoter. In these embodiments, the PXYL promoter is under the control of the tetracycline repressor (See FIG. 1).

The present invention provides methods for determining whether a particular gene of interest is important for viability of a cell under various conditions. Bacterial cells are transformed with the footprinting plasmid of the invention while being maintained in environmentally permissible conditions for plasmid replication. The transformed culture is then subjected to growth during which the regulatable promoter is activated, inducing expression of transposase which promotes insertion of the transposon (first phase). In the case of a temperature sensitive origin of replication, expression is induced at the permissive temperature for the plasmid. The bacterial culture is then grown under conditions in which both insertion of the transposon and replication of the plasmid are repressed (second phase). Again, in the case of a temperature sensitive promoter, a non-permissive temperature for the plasmid is used. Selecting for chromosomal insertions of the transposon may be achieved by selecting for the marker carried in the transposon.

After the first and second phases are completed, chromosomal DNA is extracted from an aliquot of the bacterial culture and the presence of the transposon in different genes of interest is analyzed. Preferably, PCR is used to detect transposon insertion events. PCR amplification may be performed, e.g., using sets of primers in which one primer comprises sequences from or complementary to a sequence derived from the gene of interest and a second primer comprises sequences derived from or complementary to the transposon.

The PCR products derived from samples extracted after the first and second phases are compared, analyzing both frequency and quantity. Particular genes may then be categorized as more or less important for growth, depending on the degree to which transposon insertions present in the first phase are lost in the second phase. The transposon insertions present in an open reading frame of a particular gene are characterized by the abundance and length of the PCR products derived from chromosomal DNA isolated after the first and second phases. For genes that are important for the viability or growth of the cell, the PCR products obtained from the second phase will decrease in magnitude relative to the first phase. In this embodiment of the footprinting technology, the reaction conditions for the PCR based detection have been selected such that the signals resulting from the PCR reactions are barely over the threshold of detection of the instrumentation. A gene in which at least 90% of the transposon insertions present in the first sample (T0) are depleted in the later samples (T25) is likely to be important for growth and viability (Smith et al., 1995, Proc. Natl. Acad. Sci., 92:6479).

DNA, Vectors, and Host Cells

In practicing the present invention, many conventional techniques in molecular biology, microbiology, and recombinant DNA, are used. Such techniques are well known and are explained fully in, for example, Sambrook et al., 1989, Molecular Cloning: A Laboratory Manual, Second Edition, Cold Spring Harbor Laboratory Press, Cold Spring Harbor, New York; DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol.154 and Vol.155 (Wu and Grossman, and Wu, eds., respectively).

Nucleic acids comprising any of the sequences disclosed herein or subsequences thereof can be prepared by standard methods using the nucleic acid sequence information provided. For example, DNA can be chemically synthesized using, e.g., the phosphoramidite solid support method of Matteucci et al., 1981, J. Am. Chem. Soc. 103:3185, the method of Yoo et al., 1989, J. Biol. Chem. 764:17078, or other well known methods. This can be done by sequentially linking a series of oligonucleotide cassettes comprising pairs of synthetic oligonucleotides, as described below.

Insertion of nucleic acids (typically DNAs) into a vector is easily accomplished when the termini of both the DNAs and the vector comprise compatible restriction sites. If this cannot be done, it may be necessary to modify the termini of the DNAs and/or vector by digesting back single-stranded DNA overhangs generated by restriction endonuclease cleavage to produce blunt ends, or to achieve the same result by filling in the single-stranded termini with an appropriate DNA polymerase.

Alternatively, any site desired may be produced, e.g., by ligating nucleotide sequences (linkers) onto the termini. Such linkers may comprise specific oligonucleotide sequences that have desired restriction sites. Restriction sites can also be generated by the use of PCR. (Saiki et al., 1988, Science 239:48). The cleaved vector and the DNA fragments may also be modified if required by homopolymeric tailing.

The nucleic acids may be isolated directly from cells. Alternatively, PCR can be used to produce the nucleic acids of the invention, using either chemically synthesized strands or genomic material as templates. Primers used for PCR can be synthesized using the sequence information provided herein, or information available from publicly available nucleotide sequence databases. The primers can be further designed to introduce appropriate new restriction sites to facilitate incorporation into a given vector for recombinant expression.

The nucleic acids may also be modified by many means known in the art. Non-limiting examples of such modifications include methylation, “caps,” substitution of one or more of the naturally occurring nucleotides with an analog, internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. PNAs (Protein-Nucleic-Acids) are also included. The nucleic acid may be derivatized by formation of a methyl, ethyl phosphotriester or an alkyl phosphoramidate linkage. Furthermore, the nucleic acid sequences of the present invention may also be modified with a label capable of providing a detectable signal, either directly or indirectly. Exemplary labels include radioisotopes, fluorescent molecules, biotin, and the like.

Recombinant cloning vectors will often include one or more replication systems for cloning or expression, one or more markers for selection in the host, e.g. antibiotic resistance, and one or more expression cassettes. The DNA elements may be synthesized by standard methods, isolated from natural sources, or prepared as hybrids, etc. Ligation of the transposable elements into a transcriptional regulatory elements and/or to other sequences may be achieved by known methods. Suitable host cells may be transformed/transfected/infected as appropriate by any suitable method including electroporation, CaCl₂ mediated DNA uptake, microinjection, microprojectile, or other established methods. Appropriate host cells include bacteria. Non-limiting examples of bacteria include, but are not limited to, Escherichia coli, Bacillus subtilis, Streptococcal pneumoniae, Salmonella typrimoniom, and Staphylococcus aureus.

The following examples are intended as non-limiting illustrations of the present invention.

### EXAMPLE 1

### Construction of Footprinting Plasmids

A. pG139+

PG139+(FIG. 1) contains the PXYL promoter with a tetracycline repressor operator and its negative regulator, tetR. The regulatory system PXYL-tetR was based on the tet regulatable system in pWH353 (Geissendorfer and Hillen, 1990, Appl Microbiology Biotechnology 33:657-663) derived from the vector pCP16 (Cherepanov et al., 1995, Gene 158:9-14) and provides for modulation and rapid induction of expression. Genes cloned under PXYL control are rapidly and strongly induced in the presence of anhydrotetracycline and effectively turned-off in the absence of this inducer.

(i) Temperature sensitive origin of replication for Gram-positive bacteria.

This feature allows the segregation of pG139+ from the bacterial culture at the non-permissive temperature for plasmid replication (42° C.) to produce plasmid-free cells. At permissive temperature (30° C.) normal plasmid copy number is observed. At 42° C., the plasmid behaves like a non-replicative molecule and is segregated out.

(ii) Construction-pG139+

The pG139+ footprinting plasmid carries a mini-Tn10 transposon. There is an outward reading promoter PLAC in the mini-Tn10. This promoter is included to minimize possible polar effects arising from insertion of the transposon into a group of co-regulated genes. Mini-Tn10 transposon elements are generally short (400-3000 bp). At each terminus, they carry Tn10 sequences in an inverted orientation, which flank one or more selectable markers (Kleckner et al., 1991, Methods in Enzymology 204:139). The mini-Tn10 transposon was obtained from plasmid pNK2887 catplac (PTAC-mini-Tn10-PLAC) (ATCC#77343). To construct pNK2887catplac the 1000 base pair chloramphenicol (cat) gene was PCR amplified from pC194 (Horinouch and Weisblum, 1982, J. of Bacteriology 150:815-825) using two primers directed to the end of the cat gene. The 5′primer sequence was 5′-TCGAGGATCCCCCTTATTATCAAGATAAGA-3′(SEQ ID NO:1) and the 3′primer sequence was

5′-TAATGAGTGAGAATTAATTCCAGGTTAGTGACATTAGAAAACCGACTG-3′(SEQ ID NO:2). A 120 base pair PLAC fragment was PCR amplified from pNK2887 using two primers with a 5′primer sequence of

5′-CAGTCGGTTTTCTAATGTCACTAACCTGGAATTAATTCTCACTCATTA-3′(SEQ ID NO:3) and a 3′primer sequence of 5′-ACGTCTATGGATCCTGTTTCCTGTGTGA-3′(SEQ ID NO:4). These fragments were purified using S&S Elu-Quik DNA purification kit (Schleicher & Schuell, Keene, N.H.), mixed, and subjected to a second round of PCR amplification using the 5′cat primer and the 3′PLAC primer in order to create the catplac fragment. The resulting PCR fragment was digested with BamHI and cloned into the BamHI site of pNK2887.

Plasmid pER924 (Bayles et al., 1994, Gene 147:13-20) containing an origin of replication derived from pBR322 (Balbas et al., 1986, Gene 50:3-40) and the Gram-positive temperature sensitive origin of replication from pE194 (Villafane et al., 1987, J. of Bacteriology 169:4822-4829) was digested with EcoRI. This fragment was dephosphorylated using calf intestinal phosphatase (New England Biolabs, Beverly, Md.) and ligated to the 1400 bp mini-Tn10cmplac fragment from pNK2887cmplac (Kleckner et al., 1991, Methods in Enzymology 204:139-180), that had been previously digested with EcoRI. The resulting plasmid is pG138. pNK2887cmplac was constructed using overlapping PCR gene construction to replace the Gram-negative kanamycin resistance gene in pNK2887 (ATCC#77343) with the Gram-positive chloramphenicol resistance gene from pC194. The Tn10catplac fragment was PCR amplified from pKN2887catplac with two primers. The 5′primer

(5′-TCAGGAATTCACTAGTGGTAAGGTAAACGCCATTGTCAG-3′; SEQ ID NO:5) and the 3′primer

(5′-TTAGGAATTCCTCGAGGCATAAACCAGCCATTGAGTAAG-3′; SEQ ID NO:6) had EcoRI sites at the 5′end. The resulting 1400 base pair PCR fragment containing the miniTn10catplac was digested with EcoRI and cloned into the EcoRI site of pER924 to create pG138. The PXYL-tetR system was constructed in multiple steps. The tet repressor gene was PCR amplified from pCP16 using the 5′primer (5′-GGAATTAATGATGTCTAGATTAGATAAAAGTAAAGTGATTAA-3′; SEQ ID NO:7) and the 3′primer (5′-ACCATCAGCGAAAAAGGTTATGCTGCTTTTAAGACC-3′; SEQ ID NO:8). The resulting 650 base pair fragment was linked to a Gram-positive promoter that was created by annealing two complementary primers based on the system of Geissendorfer and Hillen (1990). The 1200 base pair transposase gene was amplified from pIC333 using two primers. The 5′primer sequence was

5′-GGAACGAAAGGAGGTATATACATATGTGCGAACTCGATATTTTACAC-3′ (SEQ ID NO:9) and the 3′primer sequence was 5′-TTTTTTCAGCTGCACCTTTGGTCACCAACG-3′(SEQ ID NO:10). The 5′primer was designed to contain a Gram-positive ribosome binding site upstream of the transposase gene. The PXYL promoter was created by annealing two complementary primers based on the Geissendorfer and Hillen (1990) system. To connect the PXYL promoter with the transposase gene two framents were purifed and mixed and amplified a second time with two primers. The 5′primer sequence was 5′-GTTCATGAAAAACTAAAAAATATTGACA-3′(SEQ ID NO:11) and the 3′primer sequence was 5′-CCATCGGAAGCTGTGGTATGGCTGTGCAGG-3′(SEQ ID NO:12). The resulting 13000 base pair fragment that contains the transposase under the control of the PXYL promoter was linked to the tet repressor gene by overlapping PCR gene construction in a series of three PCR amplifications. The final product was digested with PvuII and cloned into the Smal site of pG138

Segregation experiments showed that pG139+required about 15 generations to be lost from the bacterial population when the temperature is raised to the non-permissive temperature (42° C.).

### EXAMPLE 2

### Fermentation of S. aureus Cultures Transformed with the Footprinting Plasmid

The restriction minus S. aureus strain RN4220 (Freiswirth et al., 1983, Nature 305:709-712) was transformed by electroporation with pG139+. The transformants were selected on Luria Broth (LB), which are comprised of 20 g tryptone, 10 g yeast extract and 10 g NaCl, per liter; agar plates containing 3 μg/ml erythromycin (Erm) at 30° C. The bacterial culture was inoculated from a fresh agar plate into 500 ml of warm LB (containing Erm and anhydrotetracycline (Atet) at 0.05 μg/ml) at an OD600=0.05-0.1. The 500 ml culture was incubated for approximately eight hours at 30° C. at 250 rpm, until the cells reached a density of 1×10¹⁰ cells/ml (OD600=2.5). This culture was designated T0. Samples were concentrated 1:40 for frozen glycerols and 15 ml samples were harvested for genomic DNA preparation. The remaining bacterial cell culture was centrifuged, the pellet washed, and resuspended in fresh LB.

25 mls of the culture was transferred into 500 mls of prewarmed LB supplemented with chloramphenicol (Cm) at 5 μg/ml and grown at 42° C., until the cells reached a density of 1×10⁹ cells/ml (OD600=0.9). This culture was designated T5. This cycle was repeated four more times for a total of five growth cycles. At the end of each growth cycle, bacterial cultures were harvested for genomic DNA preparation and concentrated 1:40 for frozen glycerols. In total, the five growth cycles represent approximately 25 generations.

At the end of each cycle, culture samples were withdrawn and used to estimate the extent of plasmid loss. Genomic DNA was isolated from each sample for subsequent use in PCR-ABI analysis.

Ampicillin (Amp) can be used at a final concentration of 100 μg/ml.

### EXAMPLE 3

### Fermentations To Test Essentiality of Genes in S. aureus

Twenty-five mls (10¹⁰ cells) from the T=0 culture were used to inoculate 500 ml of LB. The media was supplemented with Cm 5 μg/ml to select for transposon insertions. The culture was incubated at 42° C. until an OD₆₀₀=0.9 was reached. 25 ml of the culture were transferred to 500 ml of rich medium supplemented with Cm 5 μg/ml and the culture was fermented for a total of 25 generations by repeating this step 4-5 times. At the end of 15 and 25 generations, the cultures were harvested, resuspended in 15% glycerol at a concentration of 40:1, and frozen in aliquots for isolation of chromosomal DNA.

At the beginning of the first fermentation and at the end of 25 generations, serial dilutions were plated on selection plates (as described above for the T=0 fermentation) in order to estimate the frequency of plasmid loss. Additionally, 50 ml samples were withdrawn after 15 generations of growth (T=15) and after 25 generations (T=25). The extent of plasmid loss was determined for each sample, and genomic DNA was isolated to be footprinted.

### EXAMPLE 4

### PCR and ABI analysis of candidate genes

Conditions for PCR and primer design were similar to those previously described (Smith et al. 1995, Proc. Natl. Acad. Sci. 92:6479-6483). Primers were chosen using the program Oligo 4.06 (National Biosciences, Plymouth, Minn.) and had target sequences localized 500 to 1500 bp from the 5′ termini of each gene, except in the case for genes less than 500 bps. Primers were 24-30 bases long with calculated melting temperatures (Tm) 60-74° C. The primers were synthesized by Perkin-Elmer (Perkin-Elmer Corporation) and contained a covalently attached FAM fluorescent dye. A primer directed against the mini-Tn10 transposon was unlabeled and was complementary to a region of the IS element of Tn903 flanking the Cm resistance gene of the mini-Tn10 transposon derived from pNK2887 (Tn10-1) was

(5′-GACAAGATGTGTATCCACCTTAACTTAATG-3′) [SEQ ID NO:13].

Bacterial chromosomal DNA was isolated using Qiagen columns (Qiagen Inc, Chatsworth, Calif.). 1 μg of DNA was used per PCR reaction. PCR reactions were performed in a 50 μl final volume with 0.5 μM of each primer, 250 μM dNTP, PCR buffer (10 mM Tris-HCl (pH=8.3), 50 mM KCl, 1.5 mM MgCl₂, 0.001% gelatin), and 2 units of Platinum Taq polymerase (Life Technologies, Inc.). PCR reaction conditions were:

(i) 94° C. for 1 min;

(ii) 94° C. for 30 sec; 64° C. for 45 sec; and 72° C. for 2 min;

(iii) repeat (ii) 35 times; and

(iv) 72° C. for 10 min.

When necessary, annealing temperatures of 55° C. and 60° C. were used to increase signal strength.

For Applied Biosystems Incorporated (ABI) analysis, 3 μl of the PCR reaction was added to 3 μl of a loading solution comprised of a 1:1:5 ratio of 100 mg/ml Blue dextran: Gene Scan 2500 Tamara gene standards (Perkin-Elmer): deionized formamide. Samples were denatured for 3 min in a boiling water bath, immediately placed in an ice-water bath, and loaded (1.5-3 μl) onto an 5% ABI gel (Long Ranger, FMC) containing 6M Urea and 1×TBE buffer as recommended by the manufacturers. ABI analysis was performed in an ABI prism DNA sequencer, model 377, from Applied Biosystems (Perkin-Elmer) using the collection software for the 377XL model version 2.0, and an ABI prism GeneScan software version 2.0.2. Size and distribution of PCR products were analyzed with an ABI prism Genotyper software package version 2.0.

### EXAMPLE 5

### Analysis of Non-Essential and Essential Genes

Fresh colonies from strain RN4220 harboring the plasmid pG139+ (PXYL-Tn10) were used to inoculate 50 ml of LB supplemented with Erm. Growth of this culture for 5 generations under inducing conditions (0.05 μg/ml Atet) in rich medium, was performed as described in Example 2. At the end of growth, cells from 10 liters of culture were collected, resuspended in 15% glycerol at a concentration of 40:1, and frozen in aliquots as described in Example 2. DNA was obtained from 10 ml frozen glycerol cultures as described in Example 2. This DNA was designated T=0 DNA.

As expected, the plasmid is stably maintained in the cells throughout growth under conditions that allow replication of the plasmid.

A 2 ml frozen glycerol culture from the T=0 fermentation was thawed carefully on ice and brought to room temperature. These cells were cultured in rich medium for 25 generations under conditions that repress both transposase expression and plasmid replication, and select for transposon insertions. The cells were harvested, concentrated and frozen as described in Example 2. DNA was obtained from culture sample as described in Example 2. This DNA was designated T=15 and T=25 DNA.

PCR and ABI analysis was then performed using TN, T0, T15, and T=25 DNA as template and specific primers for the both broad spectrum and Gram-positive only essential and non-essential genes. The essential/non-essential nature of these genes has been previously determined through knockout mutagenesis. For PCR reactions, 1 μg of DNA was used with unlabeled transposon primers and the specific, labeled primers for the above genes as described in Example 4. PCR products were analyzed as described in Example 4.

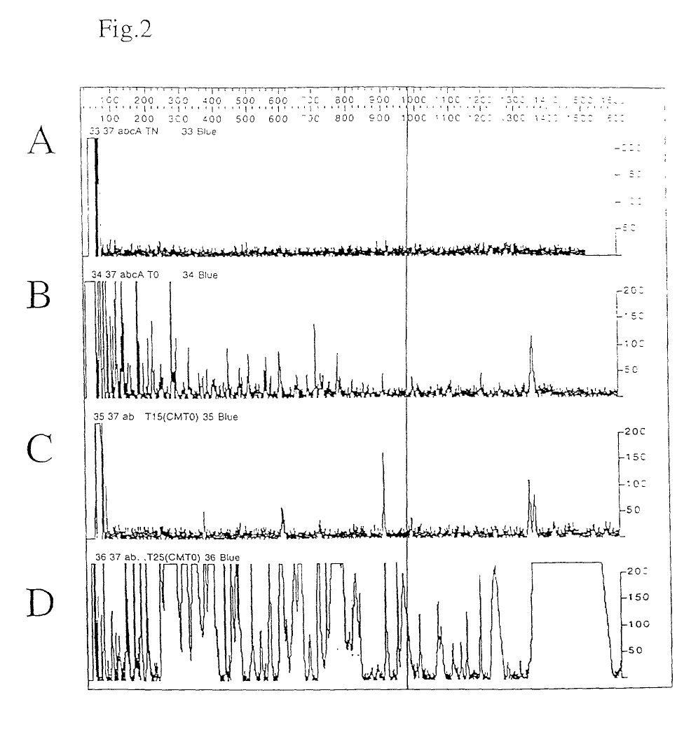

FIG. 2 shows the Tn10 insertion profiles of mutagenesis in a known nonessential gene S. aureus gene, abca. A specific primer complementary to a segment of this gene is located 989 bps downstream of the ATG translation initiation codon, in the reverse orientation. The primer sequence was 5′-ATTCTACTACTTGCACCGACTGCC-3′[SEQ ID NO:14]. The use of this primer allows a survey of the first 989 bps of the gene and approximately 600 bps of the preceding coding sequence, including the 5′regulatory region, and a segment of the upstream gene.

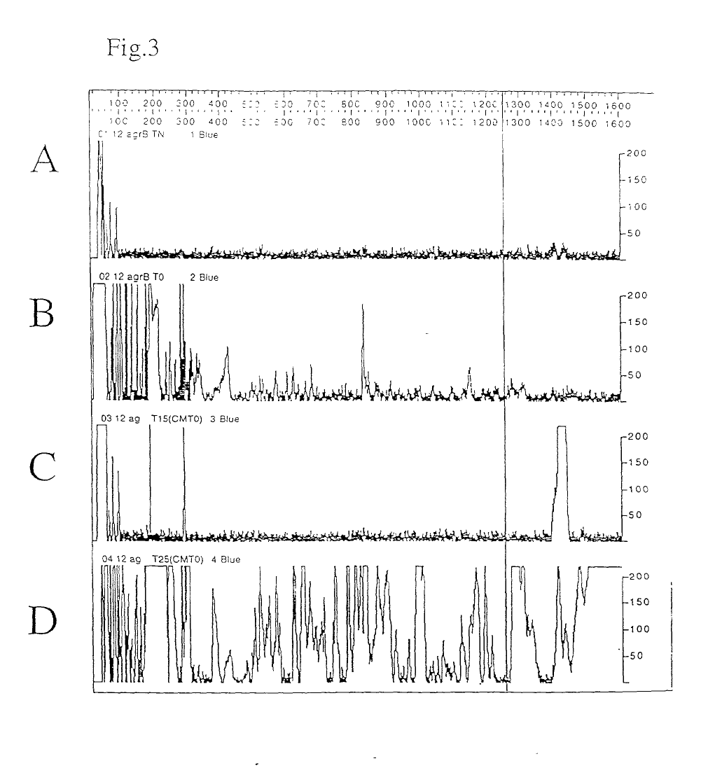

In FIG. 3, the footprinting approach is also shown for a known nonessential gene S. aureus gene, agrB. A specific primer complementary to a segment of this gene is located 1245 bps downstream of the ATG translation initiation codon, in the reverse orientation. The primer sequence was 5′-ACCACGACCTTCACCTTTAGTAGA-3′[SEQ ID NO:15]. The use of this primer allows a survey of the first 1245 bps of the gene and approximately 400 bps of the preceding coding sequence, including the 5′regulatory region, and a segment of the upstream gene.

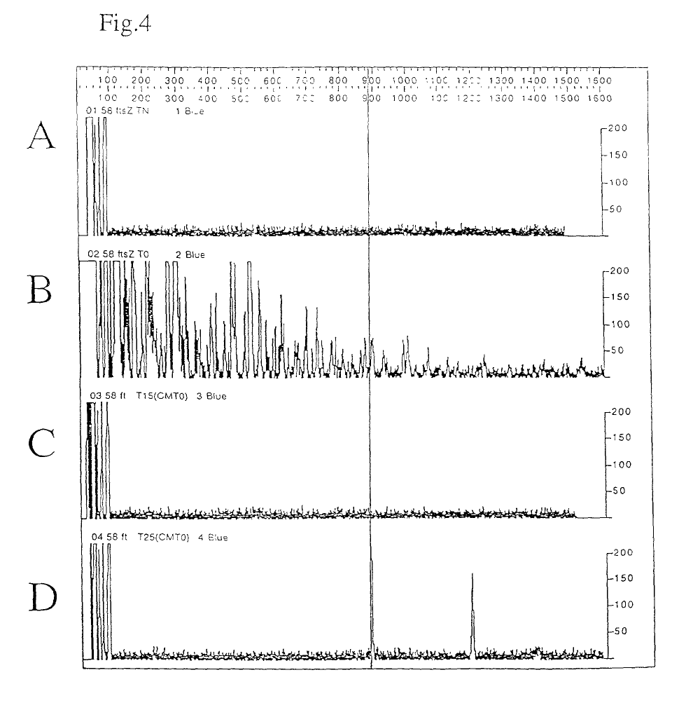

FIG. 4 shows the Tn10 insertion profiles of mutagenesis in an expected essential S. aureus gene,ftsz. A specific primer complementary to a segment of this gene is located 894 bps downstream of the ATG translation initiation codon, in the reverse orientation. The primer sequence was 5′-TGTGCTTCAAATAATGACAATGACTC-3′[SEQ ID NO:16]. The use of this primer allows a survey of the first 894 bps of the gene and approximately 700 bps of the preceding coding sequence, including the 5′regulatory region, and a segment of the upstream gene.

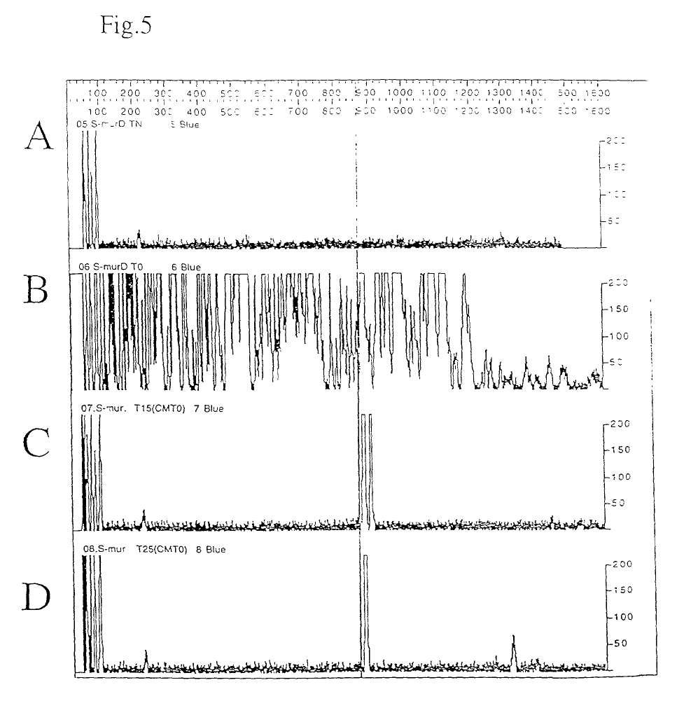

FIG. 5 shows the Tn10 insertion profiles of mutagenesis in another expected essential gene, murD. A specific primer complementary to a segment of this gene is located 885 bps downstream of the ATG translation initiation codon, in the reverse orientation. The primer sequence was 5′-CACCAGGCAATACTAGATCTTCAG-3′[SEQ ID NO:17]. The use of this primer allows a survey of the first 885 bps of the gene and approximately 900 bps of the preceding coding sequence, including the 5′regulatory region, and a segment of the upstream gene.

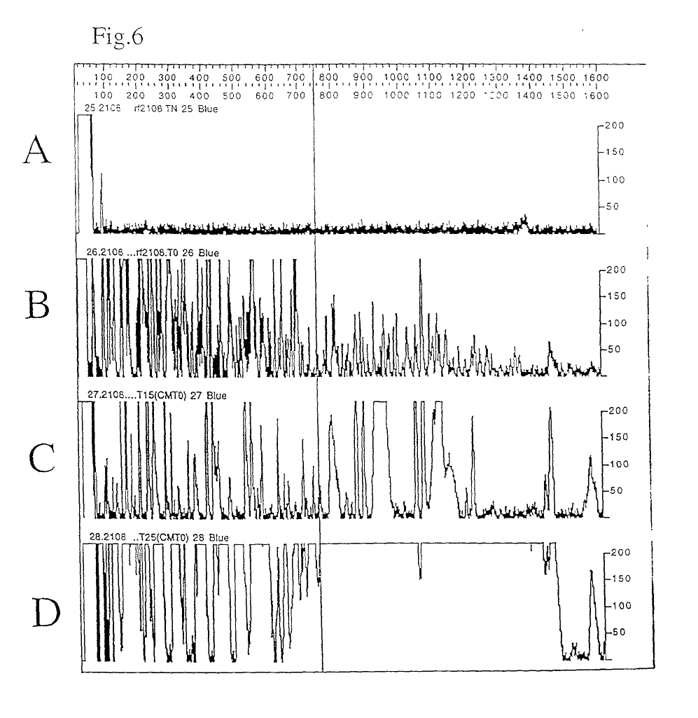

FIG. 6 shows the Tn10 insertion profiles of mutagenesis in an unknown test gene SA2108. A specific primer complementary to a segment of this gene is located 758 bps downstream of the ATG translation initiation codon, in the reverse orientation. The primer sequence was 5′-TGCAACATTTGATAACCAATGCGTGGA-3′[SEQ ID NO:18]. The use of this primer allows a survey of the first 758 bps of the gene and approximately 800 bps of the preceding coding sequence, including the 5′regulatory region, and a segment of the upstream gene. This profile indicates that 2108 is a nonessential gene.

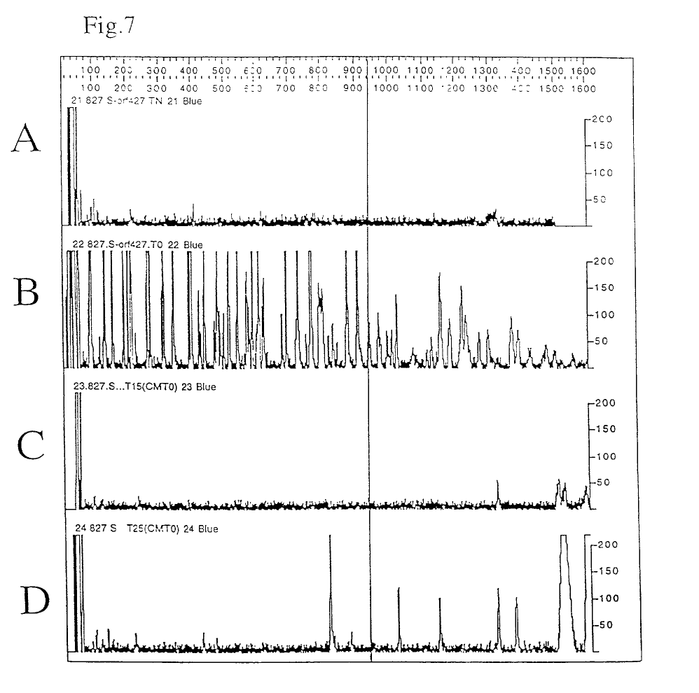

FIG. 7 shows the Tn10 insertion profiles of mutagenesis in an unknown test gene SA827. A specific primer complementary to a segment of this gene is located 968 bps downstream of the ATG translation initiation codon, in the reverse orientation. The primer sequence was 5′-TCATTCTCCTTTTTCGCTCGAGGATGT-3′[SEQ ID NO:19]. The use of this primer allows a survey of the first 968 bps of the gene and approximately 600 bps of the preceding coding sequence, including the 5′regulatory region, and a segment of the upstream gene. This profile indicates an essential gene.

Essential vs. non-essential. A comparison of the PCR profiles of the non-essential and essential genes revealed the presence of numerous insertions in all T0 samples and a similar pattern of mutagenesis, implying that T0 cells harbor random insertions. The T25 samples for the non-essential genes revealed an increase in the number of insertions in the selected population generations in rich medium (LB) containing Cm. These results indicate that these genes are dispensable for growth and are therefore non-essential genes under the conditions tested.

In contrast to the non-essential genes, the insertions observed at T=25 essential genes were no longer present in T25 DNA, which contained little specific insertions in either of the two genes. These results suggest that cells carrying insertions in these genes are unable to grow and survive in rich medium, but, rather, are lost during the second growth phase. Thus, these insertions are lost from the population and are not detected PCR amplification at T25. Based on these results, it is concluded that these genes are required for growth under the conditions tested and constitute essential genes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A plasmid comprising
(i) a transposon carrying a selectable marker gene;
(ii) a gene encoding a transposase that catalyzes insertion of said transposon into genomic DNA in vivo, wherein said gene is operably linked to a regulatable promoter; and
(iii) an environmentally sensitive bacterial origin of replication.

2. The plasmid of claim 1, wherein the environmentally sensitive bacterial origin of replication is a Gram-positive origin.

3. The plasmid of claim 2, further comprising a second Gram-negative origin of replication.

4. The plasmid of claims 1, 2 or 3, wherein the environmentally sensitive bacterial origin of replication is temperature sensitive.

5. The plasmid of claim 2, wherein said transposon is derived from Tn10; said transposase comprises Tn10 ATS; said regulatable promoter is operably linked to a PXYL promoter; said origin of replication is derived from pE194; and said selectable marker gene is a chloramphenicol resistance gene.

6. The plasmid of claim 5, further comprising a second Gram-negative origin of replication derived from pBR322.

7. A plasmid comprising
(i) a Tn10 transposon carrying a chloramphenicol resistance gene;
(ii) a gene encoding Tn10 ATS transposase operably linked to a a PXYL promoter;
(iv) a temperature sensitive bacterial origin of replication derived from pE194; and
(iv) a bacterial origin of replication derived from pBR322.

8. The plasmid of claim 7 consisting of pG139+.

9. A method for determining whether a bacterial gene of interest is important for viability of the bacterial cell from which it is derived, said method comprising:
(a) transforming bacterial cells with a plasmid as defined in claim 1;
(b) incubating said transformed cells under conditions in which said first and second regulatable promoters are active, to produce a mutagenized culture;
(c) subjecting said mutagenized culture to multiple cycles of fermentation under conditions in which episomal replication of the plasmid and genomic insertion of the transposon are repressed;
(d) extracting genomic DNA from the culture of step (c); and
(e) determining the presence or absence of the transposon in said gene of interest; wherein a gene lacking said transposon is important for viability of said bacterial cell.

10. The method of claim 9, wherein the gene of interest is essential.

11. The method of claim 9, wherein the conditions in step (c) additionally inhibits replication of the plasmid.

12. The method of claim 9, wherein said determining comprises polymerase chain reaction.

13. The method of claim 9, wherein the environmentally sensitive bacterial origin of replication is a Gram-positive origin.

14. The method of claim 13, further comprising a second Gram-negative origin of replication.

15. The method of claim 9, 13 or 14, wherein the environmentally sensitive bacterial origin of replication is temperature sensitive.

16. The method of claim 13, wherein said transposon is derived from Tn10; said transposase comprises Tn10 ATS; said regulatable promoter is operably linked to a PXYL promoter; said origin of replication is derived from pE194; said selectable marker gene is a chloramphenicol resistance gene.

17. The method of claim 16, further comprising a second Gram-negative origin of replication derived from pBR322.

18. The method of claim 9, wherein said bacterial cell is a Gram-negative bacterial cell.
